(19) United States
(12) Patent Application Publication
(10) Pub. No.: US 2023/0165522 A1
(43) Pub. Date: Jun. 1, 2023

(54) CHRONIC OBSTRUCTIVE PULMONARY DISEASE MONITORING

(71) Applicant: Medtronic, Inc., Minneapolis, MN (US)

(72) Inventor: Geert Morren, Vissenaken (BE)

(21) Appl. No.: 18/054,035

(22) Filed: Nov. 9, 2022

Related U.S. Application Data
(60) Provisional application No. 63/264,744, filed on Dec. 1, 2021.

(51) Int. Cl. A61B 5/00; A61B 5/389; A61B 5/313

(52) U.S. Cl. CPC A61B 5/4842; A61B 5/389; A61B 5/313; A61B 5/7278; A61B 5/6867; A61B 5/4561; A61B 2562/0219; A61B 5/7405; A61B 5/742; A61B 5/7455; A61B 5/486; A61B 5/0816

(57) ABSTRACT

An example device includes memory configured to store a measure of COPD severity of a patient and processing circuitry communicatively coupled to the memory. The processing circuitry is configured to receive an electromyogram (EMG) of the patient, receive one or more signals indicative of respiration rate of the patient, and receive one or more signals indicative of tidal volume of the patient. The processing circuitry is configured to determine, based on the respiration rate of the patient and the tidal volume of the patient, a minute ventilation of the patient. The processing circuitry is configured to determine, based on the minute ventilation of the patient and the EMG of the patient, the measure of COPD severity of the patient, and generate an indication for output that is based at least in part on the measure of COPD severity of the patient.

This application claims the benefit of U.S. Provisional Application No. 63/264,744, filed Dec. 1, 2021, the entirety of which is hereby incorporated by reference.

### FIELD

This disclosure generally relates to medical devices and, more particularly, patient monitoring by medical devices.

### BACKGROUND

Medical devices may be used to monitor physiological parameters of a patient. For example, some medical devices are configured to sense electrogram (EGM) signals, e.g., electrocardiogram (ECG) signals, indicative of the electrical activity of tissue, such as a heart, via electrodes.

Chronic Obstructive Pulmonary Disease (COPD) is a progressive lung disease characterized by persistent airflow limitation. The main symptom of COPD is dyspnea (e.g., breathlessness), defined as “a subjective experience of breathing discomfort that consists of qualitatively distinct sensations that vary in intensity.” In clinical practice, dyspnea is usually assessed using patient questionnaires and/or rating scales that are designed to determine the relation the severity of dyspnea and the associated level of activity, as dyspnea is most pronounced upon exertion. Although subjective, such questionnaires (e.g., (modified) Borg scale for dyspnea, Medical Research Council (MRC), Shortness of Breath with Daily Activities (SOBDA), University of California San Diego Questionnaire (UCSDQ), etc.) are valuable for prognosis or prediction of acute exacerbations of COPD.

### SUMMARY

COPD is progressive lung disease characterized by persistent airflow limitation and a gradual deterioration in lung function with multiple distressing symptoms, such as dyspnea. Although subjective, the questionnaires mentioned above may be used to evaluate disease progression over time in patients suffering from COPD.

A more objective assessment of dyspnea may be derived from the electromyogram (EMG) of respiratory muscles (e.g., intercostals and/or diaphragm). An EMG-derived metric has recently been shown to correlate well with dyspnea severity during exercise testing and to be able to predict clinical deterioration in exacerbations of COPD. See Suh E-S, Mandal S, Harding R, et. al., “Neural respiratory drive predicts clinical deterioration and safe discharge in exacerbations of COPD”, Thorax 2015; 0:1-8 doi:10.1136/thoraxjn1-2015-207188. For example, the dyspnea severity may be evaluated non-invasively by measuring an external surface EMG of the parasternal muscles or the diaphragm using electrodes positioned on the skin. Feasibility and reproducibility of this non-invasive EMG analysis outside of reference centers has not been shown, and is questioned due to the sensitivity of the measurement to, for example, electrode positioning. See Sferrazza Papa GF, De Giampaulis P, Di Marco F, Corbo M. Is neural drive the missing piece in the puzzle of COPD exacerbation? Minerva Med 2016;107 (Suppl. 1 to No. 6): 9-13. Additionally, such testing is not easily conducted during daily life of a COPD patient.

In general, this disclosure describes techniques for monitoring of disease status in a COPD patient at various levels of activity during daily life. In some examples, an implantable or insertable EMG measurement device may be employed to monitor COPD. EMG measurements made using electrodes integrated in an implantable or insertable device at a relatively fixed position, can be directly compared over longer time periods. An insertable or implantable medical device system may determine an EMG of a patient. The insertable or implantable medical device system may determine minute ventilation of the patient, for example, based on signals indicative of respiration rate and tidal volume of the patient. The insertable or implantable medical device system may determine a measure of COPD severity based on the minute ventilation and the EMG. In some examples, these techniques may be repeated over time to monitor COPD progression over time. The measure(s) of COPD severity may be output to an external device which may, for example, display the measure(s) of COPD severity to be viewed by a clinician. The displayed measure(s) of COPD severity may guide medical treatment of the patient by the clinician, which may prevent or shorten potential hospital stays of a COPD patient.

In some examples, a method comprises: receiving, by a medical device, an electromyogram (EMG) of a patient; receiving, by the medical device, one or more signals indicative of a respiration rate of the patient; receiving, by the medical device, one or more signals indicative of a tidal volume of the patient; determining, by the medical device and based on the one or more signals indicative of the respiration rate of the patient, the respiration rate of the patient; determining, by the medical device and based on the one or more signals indicative of the tidal volume of the patient, the tidal volume of the patient; determining, by the medical device and based on the respiration rate of the patient and the tidal volume of the patient, a minute ventilation of the patient; determining, by the medical device and based on the minute ventilation of the patient and the EMG of the patient, a measure of COPD severity of the patient; and generating, by the medical device, an indication for output that is based at least in part on the measure of COPD severity of the patient.

In some examples, a medical device system comprises memory configured to store a measure of COPD severity of a patient; and processing circuitry communicatively coupled to the memory, the processing circuitry being configured to: receive an electromyogram (EMG) of the patient; receive one or more signals indicative of respiration rate of the patient; receive one or more signals indicative of tidal volume of the patient; determine, based on the one or more signals indicative of the respiration rate of the patient, the respiration rate of the patient; determine, based on the one or more signals indicative of the tidal volume of the patient, the tidal volume of the patient; determine, based on the respiration rate of the patient and the tidal volume of the patient, a minute ventilation of the patient; determine, based on the minute ventilation of the patient and the EMG of the patient, the measure of COPD severity of the patient; and generate an indication for output that is based at least in part on the measure of COPD severity of the patient.

In some examples, a computer-readable medium comprising instructions, which, when executed, cause processing circuitry to: receive an electromyogram (EMG) of a patient; receive one or more signals indicative of respiration rate of the patient; receive one or more signals indicative of tidal volume of the patient; determine, based on the one or more signals indicative of the respiration rate of the patient, the respiration rate of the patient; determine, based on the one or more signals indicative of the tidal volume of the patient, the tidal volume of the patient; determine, based on the respiration rate of the patient and the tidal volume of the patient, a minute ventilation of the patient; determine, based on the minute ventilation of the patient and the EMG of the patient, the measure of COPD severity of the patient; and generate an indication for output that is based at least in part on the measure of COPD severity of the patient.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a conceptual drawing illustrating an example of a medical device system configured to monitor physiological parameters of a patient in accordance with the techniques of the disclosure.

FIG. 2 is a block diagram illustrating an example configuration of the implantable medical device (IMD) of FIG. 1.

FIG. 3 is a conceptual side-view diagram illustrating an example configuration of the IMD of FIGS. 1 and 2.

FIG. 4 is a functional block diagram illustrating an example configuration of an external device.

FIG. 5 is a graphical diagram illustrating an example of dyspnea scores taken at various minute ventilation levels over time.

FIG. 6 is a conceptual diagram illustrating an example of determining a measure of COPD severity.

FIG. 7 is a composite graphical diagram illustrating examples of lower minute ventilation and higher minute ventilation with a comparable EMG-RMS.

FIG. 8 is a flow diagram illustrating example COPD monitoring techniques of this disclosure.

Like reference characters refer to like elements throughout the figures and description.

### DETAILED DESCRIPTION

A variety of types of implantable or insertable medical devices monitor physiological parameters of a patient. Implantable (or insertable) medical devices (IMDs) can sense and monitor cardiac EGMs, and detect arrhythmia episodes. Example IMDs that monitor cardiac electrograms (EGMs) include pacemakers and implantable cardioverter-defibrillators, which may be coupled to intravascular or extravascular leads, as well as pacemakers with housings configured for implantation within the heart, which may be leadless. Some IMDs that do not provide therapy, e.g., implantable patient monitors, sense cardiac EGMs. One example of such an IMD is the Reveal LINQ™ Insertable Cardiac Monitor (ICM), available from Medtronic plc, which may be inserted subcutaneously. Such IMDs may facilitate relatively longer-term monitoring of patients during normal daily activities, and may periodically transmit collected data or alerts to external devices or to a patient monitoring service, such as the Medtronic Carelink™ Network.

In some examples, an IMD may be used to monitor a disease state or disease progression of a COPD patient. By objectively determining a measure of COPD severity during daily life and generating an indication for output that is based at least in part on the measure of COPD severity to an external device, the IMD may facilitate the treatment of the COPD patient, thereby preserving as much of the health status of the patient as possible.

As dyspnea is most pronounced during exertion (and is often the cause of exercise limitation in COPD patients), the assessment of dyspnea should also take into account the level of exertion of the patient. For example, the level of exertion of the patient may be approximated by measuring the minute ventilation (e.g., in liters/minute) of the patient. See O'Donnell et al, “Lung hyperinflation in COPD: applying physiology to clinical practice”, COPD Research and Practice (2015) 1:4. In some examples, minute ventilation may be a volume of gas inhaled (inhaled minute volume) and/or exhaled (exhaled minute volume) into or from a person's lungs per unit of time. In some examples, the unit of time is a minute. In other examples, although referred to as minute ventilation, the unit of time may be different than a minute.

The dyspnea intensity (e.g., measured using the EMG) in a COPD patient, for example, can be the same for a given COPD patient when a) the patient is performing heavier exercises (e.g., with a higher minute ventilation) in a better health condition and b) the patient is performing light exercises (e.g., with a smaller minute ventilation) in a worsened health condition. Therefore, during cardiopulmonary exercise testing at a facility, minute ventilation may be monitored using sensors measuring the volume of inhaled/exhaled air via a patient worn facemask simultaneously with the dyspnea intensity determination while the patient exercises on a stationary bicycle at different intensity levels. Because this testing cannot be easily performed at home, the simultaneous assessment of EMG and minute ventilation during daily life (e.g., over a longer time period) is in practice limited to EMG measurements in a resting condition assuming that the minute ventilation in this condition is always the same. This limits the current methodology to testing during resting conditions at home, whereas dyspnea is most pronounced during activities or exercise, or to testing in cardiopulmonary testing facilities.

However, there are limitations of the existing techniques to assess dyspnea using an EMG which prevent or limit the use of the techniques to monitor dyspnea and/or COPD severity during daily life (e.g., outside of a cardiopulmonary testing facility and over longer time periods). Due to the sensitivity to electrode positioning, absolute values of the EMG, even when taken in a cardiopulmonary testing facility, cannot be compared over longer time periods as the electrodes used to take the EMG may be repositioned or reapplied. Therefore, at such cardiopulmonary testing facilities, the patients are asked to make a ‘reproducible maximal sniff maneuver,’ and the ratio of the resting EMG metric to the EMG measured during the maximal sniff maneuver is then used as a marker to monitor change in disease status or disease progression. There may be problems with this proposed normalization of the EMG. First, a patient may be likely to perform a different maximal sniff maneuver every time the patient performs the maximal sniff maneuver (e.g., the patient may not exactly recruit the same motor units to the same extent). Second, the procedure does not completely suppress the influence of the electrode positions. Because additional motor units are recruited during the maximal sniff maneuver, the position of the electrodes relative to the motor units may still yield a different ratio of EMG amplitudes even if all other conditions remain the same.

In general, this disclosure describes techniques for monitoring of disease status in a COPD patient at various levels of activity during daily life by overcoming the above-mentioned problems. For example, this disclosure describes a medical device system that uses processing circuitry to determine a measure of COPD severity in a COPD patient and generate an indication for output that is based at least in part on the measure of COPD severity to an external device. For example, the indication may include the measure(s) of COPD severity, physiological parameters of a patient, medical instructions (e.g., instructions for treatment, such as use an inhaler, or to seek medical attention), an alert, or the like. Such an indication may be output to an external device which may, for example, display the indication to be viewed by a clinician, the patient, or a caregiver. The displayed indication may guide medical treatment of the patient by the clinician or may prompt the patient to seek medical attention, which may prevent or shorten potential hospital stays of a COPD patient.

The medical device system may include a medical device, such as one of the devices described above or any other type of implantable device, such as a subcutaneous cardiac monitoring device, a single chamber ICD, an extravascular ICD, a subcutaneous ICD, or any other type of device configured to monitor physiological parameters of a patient. While the techniques of this disclosure are primarily described as monitoring a disease state or disease progression of a COPD patient, the techniques of this disclosure may be used to monitor other diseases or disorders. For example, the techniques of this disclosure may be used to monitor the disease state or disease progression of heart disease, heart failure, cardiopulmonary disease, sleep apnea, or other cardiac or respiratory conditions.

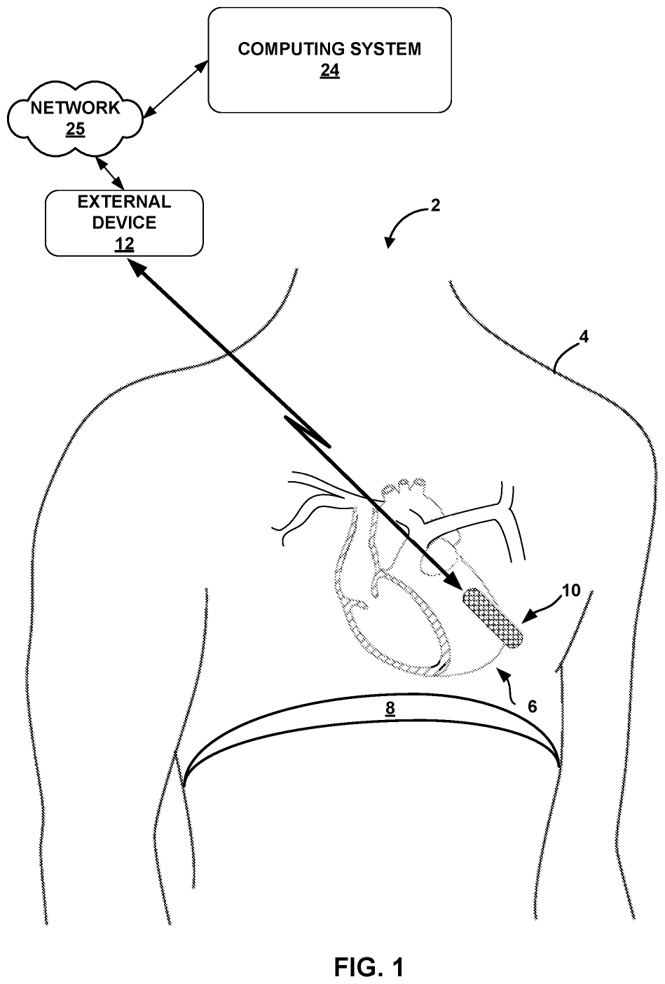

FIG. 1 is a conceptual drawing illustrating an example of a medical device system configured to monitor physiological parameters of a patient in accordance with the techniques of the disclosure. Additionally, the techniques described herein as being performed by IMD 10 may be performed by other implantable or insertable medical devices.

The example techniques may be used with IMD 10, which may be in wireless communication with an external device 12. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. In some examples, IMD 10 is positioned near the respiratory muscles (e.g., diaphragm 8, parasternal muscles, or intercostal muscles) of patient 4. For example, IMD 10 may be positioned proximate to rib 6, rib 7, or rib 8 of patient 4. IMD 10 includes a plurality of electrodes (not shown in FIG. 1), and may be configured to sense or measure any combination of a cardiac EGM (e.g., an ECG) via the plurality of electrodes, accelerometer signals, a non-cardiac EMG via the plurality of electrodes, impedance of tissue via the plurality of electrodes, optical sensor signals, temperature sensor signals, or other signals indicative of a physiological parameter of patient 4. In some examples, IMD 10 takes the form of the LINQ™ ICM. Although described primarily in the context of examples in which the medical device that monitors for COPD disease state or COPD disease progression takes the form of an ICM, the techniques of this disclosure may be implemented in systems including any one or more implantable or external medical devices, including monitors, pacemakers, defibrillators, or other devices configured to monitor physiological parameters of a patient.

External device 12 is a computing device configured for wireless communication with IMB 10. External device 12 may be, as examples, a mobile telephone or other computing device of patient 4 or another user, or a computing device configured to communicate with IMB 10. External device 12 may be configured to communicate with computing system 24 via network 25. In some examples, external device 12 may provide a user interface and allow a user to interact with IMD 10. Computing system 24 may comprise computing devices configured to allow a user to interact with IMD 10, or data collected from IMD 10, via network 25.

External device 12 may be used to receive or retrieve data from IMD 10 and may transmit the data to computing system 24 via network 25. The retrieved data may include values of physiological parameters received, measured or otherwise determined by IMD 10, measures of COPD severity, or other maladies detected by IMD 10, and physiological signals (e.g., EMG, ECG, sensor signals) recorded by IMD 10.

In some examples, computing system 24 includes one or more handheld computing devices, computer workstations, servers or other networked computing devices. In some examples, computing system 24 may include one or more devices, including processing circuitry and storage devices, that implement a monitoring system 450. Computing system 24, network 25, and monitoring system 450 may be implemented by the Medtronic Carelink™ Network or other patient monitoring system, in some examples.

Network 25 may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Network 25 may include one or more networks administered by service providers, and may thus form part of a large-scale public network infrastructure, e.g., the Internet. Network 25 may provide computing devices, such as computing system 24 and IMD 10, access to the Internet, and may provide a communication framework that allows the computing devices to communicate with one another. In some examples, network 25 may be a private network that provides a communication framework that allows computing system 24, IMD 10, and/or external device 12 to communicate with one another but isolates one or more of computing system 24, IMD 10, or external device 12 from devices external to network 25 for security purposes. In some examples, the communications between computing system 24, IMD 10, and external device 12 are encrypted.

Processing circuitry of medical device system 2 (e.g., of IMD 10, external device 12, computing system 24, and/or of one or more other computing devices) may be configured to perform the example techniques of this disclosure for monitoring for and determining an exacerbation of COPD symptoms in patient 4.

According to the techniques of this disclosure, IMD 10 may be configured to monitor a disease state of a COPD patient during daily life. IMD 10 may receive an EMG of patient 4. IMD 10 may receive one or more signals indicative of respiration rate of patient 4. IMD 10 may receive one or more signals indicative of tidal volume of patient 4. IMD 10 may determine, based on the one or more signals indicative of the respiration rate of patient 4, the respiration rate of patient 4. IMD 10 may determine, based on the one or more signals indicative of the tidal volume of patient 4, the tidal volume of patient 4. IMD 10 may determine, based on the respiration rate of patient 4 and the tidal volume of patient 4, a minute ventilation of patient 4. IMD 10 may determine, based on the minute ventilation of patient 4 and the EMG of patient 4, the measure of COPD severity of patient 4, and generate an indication for output that is based at least in part on the measure of COPD severity of patient 4.

In some examples, IMD 10 is configured to be inserted or implanted subcutaneously to ensure relatively stable positioning, and includes two or more electrodes which may be configured to sense an EMG of patient 4. In some examples, IMD 10 is configured to sample the EMG signal at a sampling rate of approximately 1000 Hz, as the EMG may contain frequencies up to approximately 500 Hz, or twice the highest frequency of the EMG. IMD 10 may be configured with a higher sensitivity or resolution than a device configured to solely monitor ECGs because the EMG may have a smaller amplitude than an ECG. In some examples, the interelectrode distance of the electrodes may be in the range of 15-80 mm. In some examples, IMD 10 may be inserted or implanted in patient 4 so as to be positioned close to the respiratory muscles, such as the parasternal, intercostal muscles or diaphragm 8, to reduced crosstalk from adjacent muscles (when compared to surface electrodes). In some examples, IMD 10 may be positioned with electrodes facing inwards, towards the core of patient 4 to enhance EMG measurements when compared to being positioned with the electrodes facing outwards, away from the core of patient 4.

For example, IMD 10 may monitor an EMG of respiratory muscles, as an objective measure of dyspnea. IMD 10 may also monitor one or more signals indicative of respiration rate and one or more signals indicative of tidal volume. IMD 10 may determine minute ventilation of patient 4 based on the respiration rate and the tidal volume. For example, IMD 10 may multiply the respiration rate by the tidal volume to determine the minute ventilation of patient 4. IMD 10 may determine, based on the minute ventilation and the EMG, a measure of COPD severity. IMD 10 may generate an indication for output that is based at least in part on the measure of COPD severity. For example, the indication may include the measure(s) of COPD severity, physiological parameters of a patient, medical instructions (e.g., instructions for treatment, such as use an inhaler, or to seek medical attention), an alert, or the like.

IMD 10 may output the indication to, for example, external device 12. In some examples, external device 12 may display the indication via a user interface. In some examples, external device 12 may transmit the indication via network 25 to computing system 24. In some examples, computing system 24 may issue an alert, such as an email, a text message, a phone call, or other alert, to a clinician to inform the clinician of the receipt of the indication, such as a measure of COPD severity, or to inform the clinician of the measure of COPD severity itself. In some examples, IMD 10 may send a message based at least in part on the measure of COPD severity of the patient to a remote computing device, such as external device 12. In some examples, IMD 10 may output for display the indication based at least in part on the measure of COPD severity of the patient. In some examples, IMD 10 may output one or more of an audible or haptic indication based at least in part on the measure of COPD severity of the patient. In some examples, IMD 10 may annotate or store data that is based at least in part on the measure of COPD severity of the patient. In some examples, IMD 10 may output a recommendation for change in patient behavior to mitigate COPD severity. In some examples, IMD 10 may output a recommendation for treatment of the patient to mitigate COPD severity.

Minute ventilation may be a surrogate for a measure of exertion. There are several possible techniques which IMD 10 may utilize to assess minute ventilation using either sensors (which may be included in IMD 10 or external to IMD 10) or deriving minute ventilation from the same biopotential measurements as the EMG. As the level of dyspnea increases for a given minute ventilation with worsening COPD disease, an absolute value of minute ventilation is not necessary. Rather relative changes in minute ventilation over time may suffice. Minute ventilation is typically calculated as the product of the tidal volume (ml/breath) and respiration rate (breaths/min) (e.g., tidal volume x respiration rate). In some examples, rather than respiration rate and/or changes in tidal volume, heart rate or pulse rate may be used as a surrogate for exertion.

In one example, IMD 10 may use impedance measurements of diaphragm 8, respiratory muscles, subcutaneous tissue surrounding IMD 10, or other tissue, to determine minute ventilation. For example, IMD 10 may stimulate tissue via one or more electrodes using a current or voltage and sense via one or more electrodes a respective resulting voltage or current. IMD 10 may use the sensed resulting current or voltage to determine the impedance of target tissue. This may be accomplished by stimulating at a single frequency, e.g., 8 kHz, and sampling at sufficiently high sampling rate (at least 2 the maximum respiration rate, e.g., 4 Hz) to generate a signal from which the minute ventilation can be derived. For example, IMD 10 may determine zero crossings of the signal, count the number of zero crossings in a period of time and divide by the length of the period of time to determine a respiration rate. The area above (or area below) the curve associated with the zero crossings may be indicative of tidal volume. Additionally, the amplitude of the impedance signal (maximum—minimum) may also be indicative of tidal volume. To assess tidal volume, IMD 10 may also take into account the posture of the subject which IMD 10 may derive from accelerometer signals. Further details regarding determining respiration parameters from an impedance signal may be found in U.S. Patent Publication No. 2020-0397308 A1, published on Dec. 24, 2020, entitled “SENSING VENTILATION PARMATERS BASED ON AN IMPEDANCE SIGNAL,” the entire content of which is hereby incorporated by reference.

In another example, IMD 10 may use signals from a 3D accelerometer signal to determine minute ventilation. The rotational movement of the chest/ribs associated with respiration induces a periodic change in the gravitational component of the accelerometer signal (e.g., due to the change in ‘tilt angle’, the angle with respect to the vertical axis). The respiration rate can be derived from the periodicity of the respiratory component. The amplitude of the respiratory component is correlated with the amplitude of the rib movement and thus to the tidal volume. To assess the tidal volume, the posture of the subject may be taken into account, which can be accomplished using the same accelerometer. For example, IMD 10 may determine a posture of patient 4 using the accelerometer signal. In some examples, the device may be positioned proximal to the lower ribs (6-8) as the rotational movement of these ribs is larger than other locations during respiration and both an intercostal and a diaphragm EMG may be measured from that location.

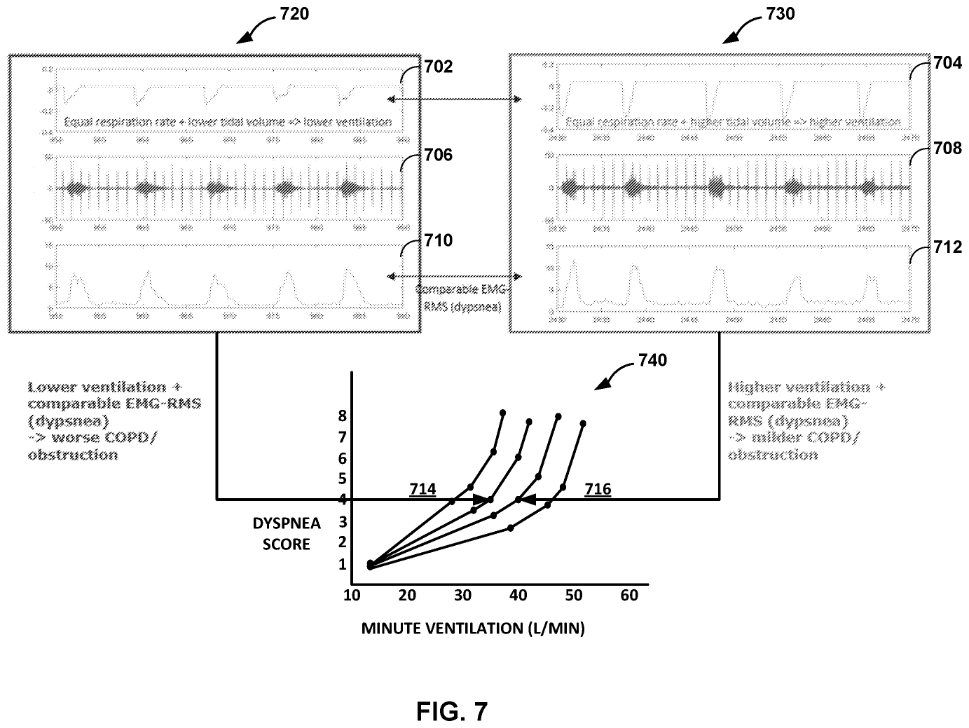

In another example, IMD 10 may use an ECG to determine minute ventilation. For example, ventilation (e.g., the mechanical effect of respiration) can be derived from a biopotential signal itself (such as an EMG or ECG). With respect to using an ECG, the amplitude of the R-peaks is affected by the respiration due to the changes in relative position of the electrodes with respect to the heart induced by the breathing. This effect, known as ECG-derived respiration (EDR), can be observed in the filtered EMG signals shown in FIG. 7. For example, the height of the R-peaks (the sharp spikes approximately every second) decreases markedly during inspiration or inhalation. In this example, neither an accelerometer nor an impedance measurement is required. In this example, IMD 10 may obtain all information needed to determine minute ventilation from a single ECG signal. In this example, the amplitude of the R-peak may be determined prior to calculating an EMG root mean squared value. The disadvantage is that the correlation of the EDR-amplitude with the tidal volume (e.g., displacement) may not be as accurate as with more direct metrics of the mechanical effect like the using the accelerometer or the impedance measurement.

In other examples, non-invasive techniques could be used to assess minute ventilation, in combination with an implantable EMG measurement, to monitor dyspnea. One such technique may include using respiratory inductive plethysmography (RIP), requiring a chest strap, however stable positioning of the chest strap may be less likely for long-term monitoring.

Another technique may be to use spirometry to measure the volume and/or flow of inhaled air. This may provide the most accurate metric of the mechanical effect, but cannot be performed continuously (upon exertion or dyspnea, patient 4 could perform spirometry and simultaneously trigger the EMG measurement). Spirometry requires a spirometer. Such a spirometer may be configured to communicate with IMD 10 either directly or via another device, such as external device 12. For example, if patient 4 felt worse on a particular day than the previous day, patient 4 could blow into the spirometer while triggering the EMG measurement of IMD 10 via external device 12. In some examples, patient 4 may blow into the spirometer a plurality of times while triggering the EMG measurement.

In another example, IMD 10 may use a pulse oximeter sensor to sense an oxygen saturation of patient 4, which may be used by IMD 10 as a surrogate for exertion. For example, the amount of oxygen in the blood of patient 4 may decrease when patient 4 is exercising. Thus, a relative amount of oxygen in the blood may be representative of a level of exertion being undertaken by patient 4.

The techniques of this disclosure may include collecting objective data on COPD symptoms multiple times during daily life under various conditions. Such objective data may be used to monitor disease progression of a COPD patient more reliably than using the maximal sniff maneuver discussed above. Additionally, because the electrodes of IMD 10 may be less likely to move and very unlikely to be removed and reattached, there is not a need to perform the maximal sniff maneuver and IMD 10 may yield more accurate or consistent measures of COPD severity over time.

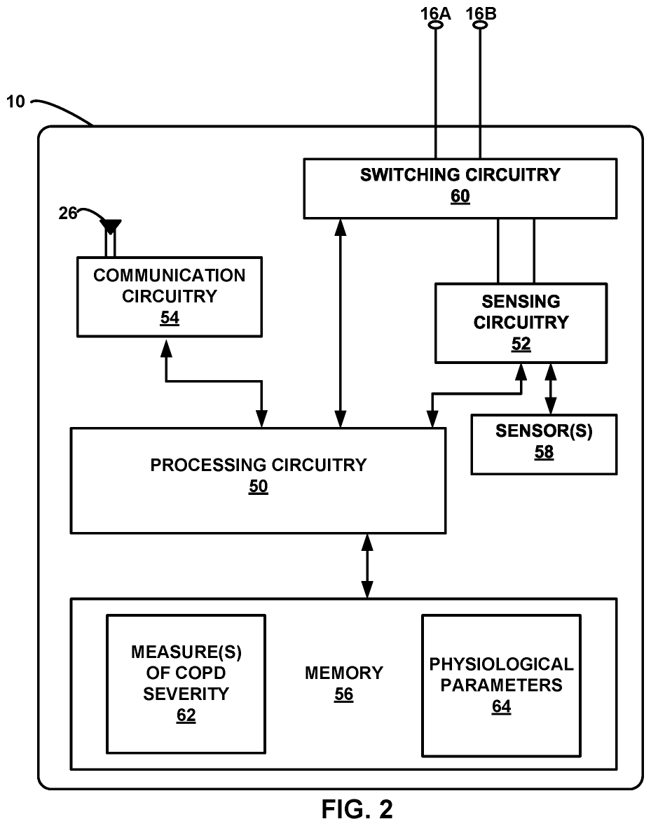

FIG. 2 is a block diagram illustrating an example configuration of IMD 10 of FIG. 1. As shown in FIG. 2, IMD 10 includes processing circuitry 50, sensing circuitry 52, communication circuitry 54, memory 56, sensors 58, switching circuitry 60, and electrodes 16A, 16B (hereinafter “electrodes 16”), one or more of which may be disposed on a housing of IMD 10. In some examples, memory 56 includes computer-readable instructions that, when executed by processing circuitry 50, cause IMD 10 and processing circuitry 50 to perform various functions attributed herein to IMD 10 and processing circuitry 50. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media. Memory 56 may also store measure(s) of COPD severity 62 and physiological parameters 64. Measure(s) of COPD severity 62 may include one or more measures of COPD severity. Physiological parameters 64 may include signals such as EMG signals, signals indicative of respiration rate, and signals indicative of tidal volume. In addition to, or alternatively, physiological parameters 64 may include determined dyspnea scores, determined respiration rates, determined tidal volumes, and/or determined minute ventilations.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16A, 16B via switching circuitry 60 as controlled by processing circuitry 50. Sensing circuitry 52 may monitor signals from electrodes 16A, 16B in order to monitor various physiological parameters of patient 4 of FIG. 1. Processing circuitry 50 may control communication circuitry 54 to output indication(s) that are based at least in part on the measure(s) of COPD severity 62 to external device 12. In some examples, processing circuitry 50 may control sensing circuitry 52 and or sensors 58 (which may include an accelerometer, an optical sensor, or other sensors) to sense or determine an EMG of patient 4, one or more signals indicative of respiration rate, and one or more signals indicative of tidal volume. Processing circuitry 50 may process such signals to determine minute ventilation of patient 4, and dyspnea score of patient 4. Processing circuitry 50 may determine a measure of COPD severity based on the minute ventilation and the dyspnea score. Processing circuitry 50 may store the measure of COPD severity in measure(s) of COPD severity 62 and may control communication circuitry 54. Processing circuitry 50 may generate an indication for output that is based at least in part on the measure of COPD severity. In some examples, processing circuitry 50 may also control communication circuitry 54 to output the indication and/or other information, such as physiological parameters 64, for example, to guide a clinician in treatment of patient 4. For example, the clinician may further assess the (individual) physiological parameters. For example, if a dyspnea score has worsened, a clinician may prescribe a different inhaler or steroids for patient 4.

Sensing circuitry 52 and/or processing circuitry 50 may be configured to process received signals, such as the EMG signal, the one or more signals indicative of respiration rate, and/or the one or more signals indicative of tidal volume to determine the physiological parameters (e.g., dyspnea score, respiration rate, tidal volume, minute ventilation) and may include filters, peak detectors, envelope calculations, in some examples.

In some examples, IMD 10 includes one or more sensors 58, such as one or more accelerometers, microphones, optical sensors, temperature sensors, pressure sensors and/or other sensors. In some examples, sensing circuitry 52 may include one or more filters and amplifiers for filtering and amplifying signals received from one or more of electrodes 16A, 16B and/or other sensors 58. In some examples, sensing circuitry 52 and/or processing circuitry 50 may include a rectifier, filter and/or amplifier, a sense amplifier, comparator, and/or analog-to-digital converter. Processing circuitry 50 may determine values of physiological parameters of patient 4 based on signals from sensors 58, which may be used to determine a measure of COPD severity of patient 4.

Communication circuitry 54 may include any suitable hardware, firmware, software or any combination thereof for communicating with another device, such as external device 12. Communication circuitry 54 may be configured to communicate using any of a variety of wireless communication schemes, such as Bluetooth® or Bluetooth Low Energy®. Under the control of processing circuitry 50, communication circuitry 54 may receive downlink telemetry from, as well as send uplink telemetry to, external device 12 or another device with the aid of an internal or external antenna, e.g., antenna 26. In some examples, processing circuitry 50 may communicate with a networked computing device (e.g., computing system 24) via an external device (e.g., external device 12) and a computer network, such as the Medtronic CareLink® Network developed by Medtronic, plc, of Dublin, Ireland.

Although described herein in the context of example IMD 10, the techniques for monitoring a disease state of COPD or a disease progression of COPD disclosed herein may be used with other types of devices. For example, the techniques may be implemented with an extra-cardiac defibrillator coupled to electrodes outside of the cardiovascular system, a transcatheter pacemaker configured for implantation within the heart, such as the Micra™ transcatheter pacing system commercially available from Medtronic PLC of Dublin Ireland, an insertable cardiac monitor, such as the Reveal LINQ™ ICM, also commercially available from Medtronic PLC, a neurostimulator, a drug delivery device, a medical device external to patient 4, a wearable device such as a wearable cardioverter defibrillator, a fitness tracker, or other wearable device, a mobile device, such as a mobile phone, a “smart” phone, a laptop, a tablet computer, a personal digital assistant (PDA), or “smart” apparel such as “smart” glasses, a “smart” patch, a “smart” watch, external device 12, or computing system 24 (both of FIG. 1). In some examples, the techniques described herein may be implemented by a combination of devices. For example, processing circuitry 50 may perform some of the techniques, while processing circuitry of one or more other devices may perform other of the techniques.

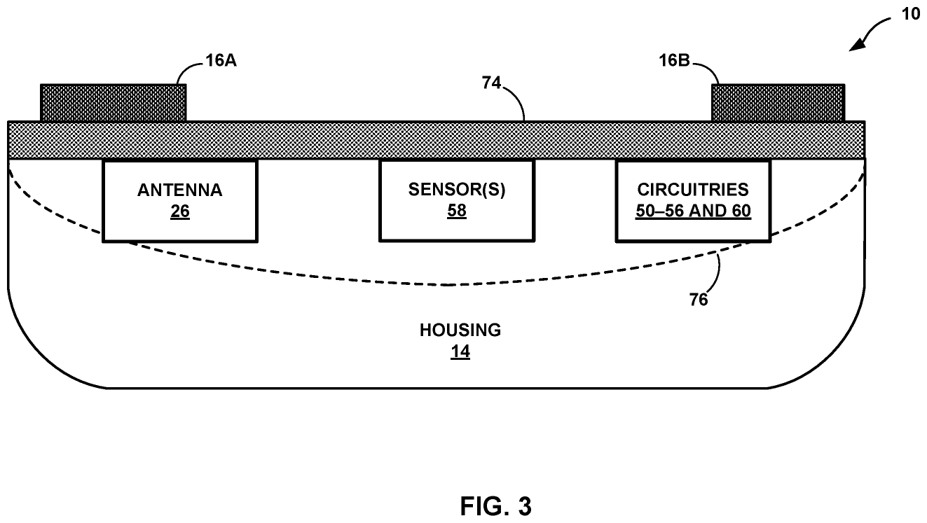

FIG. 3 is a conceptual side-view diagram illustrating an example configuration of IMD 10. In the example shown in FIG. 3, IMD 10 may include a leadless, subcutaneously-implantable monitoring device having a housing 14 and an insulative cover 74. Electrode 16A and electrode 16B may be formed or placed on an outer surface of cover 74. Circuitries 50-56 and 60, described above with respect to FIG. 2, may be formed or placed on an inner surface of cover 74, or within housing 14. In the illustrated example, antenna 26 is formed or placed on the inner surface of cover 74, but may be formed or placed on the outer surface in some examples. Sensors 58 may also be formed or placed on the inner or outer surface of cover 74 in some examples. In some examples, insulative cover 74 may be positioned over an open housing 14 such that housing 14 and cover 74 enclose antenna 26, sensors 58, and circuitries 50-56 and 60, and protect the antenna and circuitries from fluids such as body fluids.

One or more of antenna 26, sensors 58, or circuitries 50-56 may be formed on insulative cover 74, such as by using flip-chip technology. Insulative cover 74 may be flipped onto a housing 14. When flipped and placed onto housing 14, the components of IMD 10 formed on the inner side of insulative cover 74 may be positioned in a gap 76 defined by housing 14. Electrodes 16 may be electrically connected to switching circuitry 60 through one or more vias (not shown) formed through insulative cover 74. Insulative cover 74 may be formed of sapphire (i.e., corundum), glass, parylene, and/or any other suitable insulating material. Housing 14 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

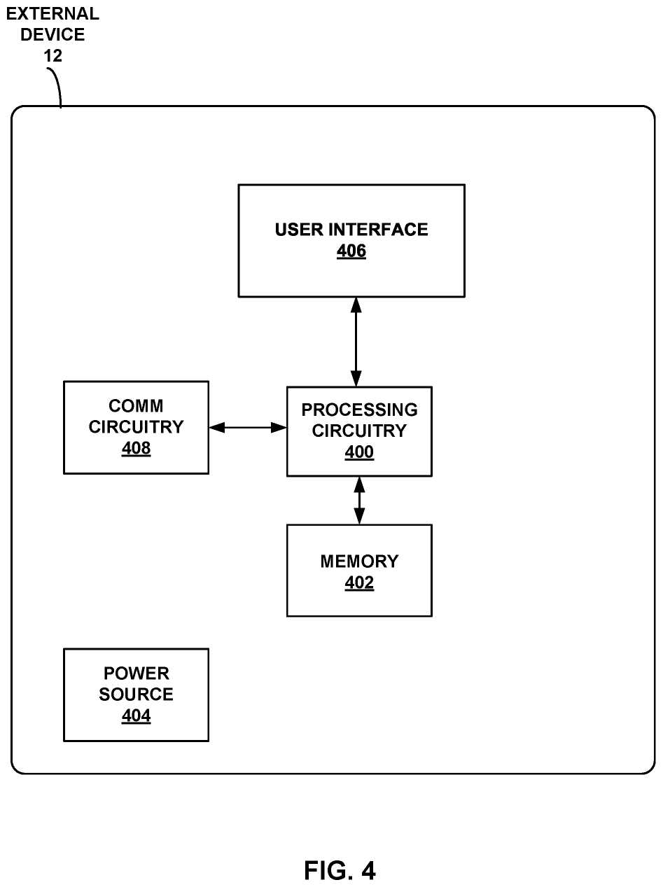

FIG. 4 is a functional block diagram illustrating an example configuration of an external device. External device 12 may include processing circuitry 400, memory 402, communication circuitry 408, user interface 406, and power source 404. Processing circuitry 400 controls user interface 406 and communication circuitry 408, and stores and retrieves information and instructions to and from memory 402. External device 21 may be configured for use as a clinician programmer or a patient programmer. Processing circuitry 400 may include any combination of one or more processors including one or more microprocessors, DSPs, ASICs, FPGAs, or other equivalent integrated or discrete logic circuitry. Accordingly, processing circuitry 400 may include any suitable structure, whether in hardware, software, firmware, or any combination thereof, to perform the functions ascribed herein to processing circuitry 400.

A user, such as a clinician or patient 4, may interact with external device 12 through user interface 406. User interface 406 may include a display, such as an LCD or LED display or other type of screen, one or more speakers, a haptic device, or other devices to present information. In some examples, user interface 406 may be configured to display, or auditorily present, to patient 4, a caregiver, or a clinician a measure of COPD severity, such that the clinician may take the appropriate measures to treat patient 4. In addition, user interface 406 may include an input mechanism to receive input from the user. The input mechanisms may include, for example, buttons, a keypad (e.g., an alphanumeric keypad), a peripheral pointing device, one or more microphones, or another input mechanism that allows the user to navigate through user interfaces presented by processing circuitry 400 of external device 21 and provide input.

In some examples, at least some of the techniques described herein may be implemented by processing circuitry 400 of external device 12. For example, processing circuitry 400 may determine the measure of COPD severity based on information received from IMD 10, generate the indication for output that is based at least in part on the measure of COPD severity, and output the indication via user interface 406.

Memory 402 may include instructions for operating user interface 406 and communication circuitry 408, and for managing power source 404. Memory 402 may also store any data retrieved from IMD 10. The clinician may use this data to determine the progression of the patient condition in order to determine future treatment. Memory 402 may include any volatile or nonvolatile memory, such as RAM, ROM, EEPROM or flash memory. Memory 402 may also include a removable memory portion that may be used to provide memory updates or increases in memory capacities. A removable memory may also allow sensitive patient data to be removed before external device 12 is used by a different patient.

Wireless telemetry in external device 12 may be accomplished by use of communication circuitry 408, which may communicate with a proprietary protocol or industry-standard protocol such as using the Bluetooth® specification set. Accordingly, communication circuitry 408 may be similar to the communication circuitry contained within by IMD 10. In alternative examples, external device 12 may be capable of infrared communication or direct communication through a wired connection. In this manner, other external devices may be capable of communicating with external device 12 without needing to establish a secure wireless connection.

Power source 404 may deliver operating power to the components of external device 12. Power source 404 may include a battery and a power generation circuit to produce the operating power. In some examples, the battery may be rechargeable to allow extended operation. Recharging may be accomplished by electrically coupling power source 404 to a cradle or plug that is connected to an alternating current (AC) outlet. In addition, recharging may be accomplished through proximal inductive interaction between an external charger and an inductive charging coil within external device 12. In other examples, traditional batteries (e.g., nickel cadmium or lithium-ion batteries) may be used. In addition, external device 12 may be directly coupled to an alternating current outlet to operate. Power source 404 may include circuitry to monitor power remaining within a battery. In this manner, user interface 406 may provide a current battery level indicator or low battery level indicator when the battery needs to be replaced or recharged. In some cases, power source 404 may be capable of estimating the remaining time of operation using the current battery.

External device 12 may receive from IMD 10 a measure of COPD severity of patient 4. For example, external device 12 may provide such information to a clinician or other device to help guide treatment of patient 4.

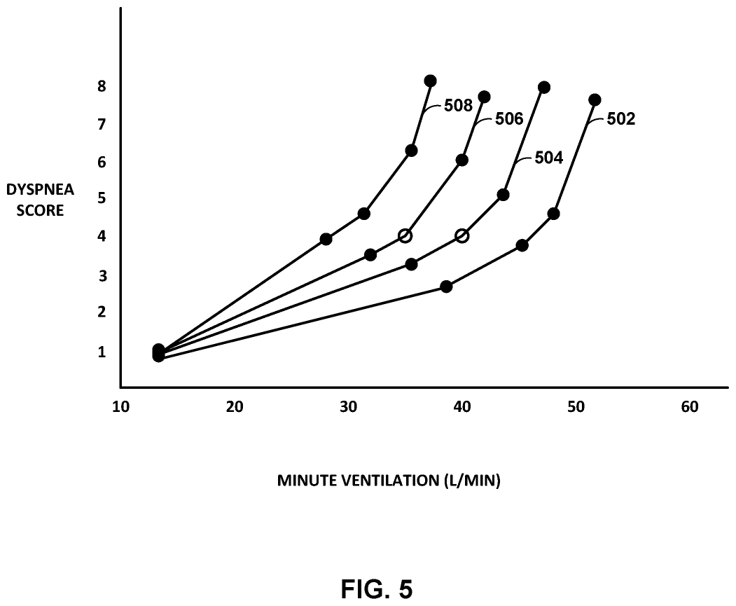

FIG. 5 is a graphical diagram illustrating an example of dyspnea scores taken at various minute ventilation levels over time. In the example of FIG. 5, a number of dyspnea scores of a patient (e.g., patient 4 of FIG. 1) are depicted over time at different minute ventilation levels. Line 502 is a representation of dyspnea scores at different minute ventilation levels (e.g., indicative of exertion or exercise levels) of patient 4 during a first time. Line 504 is a representation of dyspnea scores at different minute ventilation levels of patient 4 during a second time, later than the first time. In some examples, the difference between the first time and the second time may be on the order of weeks, months, years, etc. Line 506 is a representation of dyspnea scores at different minute ventilation levels of patient 4 during a third time, later than the second time. In some examples, the difference between the second time and the third time may be on the order of weeks, months, years, etc. Line 508 is a representation of dyspnea scores at different minute ventilation levels of patient 4 during a fourth time, later than the third time. In some examples, the difference between the third time and the fourth time may be on the order of weeks, months, years, etc. The circles on lines 502-508 represent dyspnea scores at a given minute ventilation level. As can be seen by examining the two open or unfilled circles, patient 4 may have a same dyspnea score (e.g., approximately 4) during the second time (line 504) as during the third time (line 506) at a higher minute ventilation, e.g., approximately 40 L/min, than during the third time (e.g., approximately 35 L/min). Thus, patient 4 is having the same amount of breathlessness during lighter exertion or exercise during the third time than during the second time. This represents that the disease state of patient 4 is worsening.

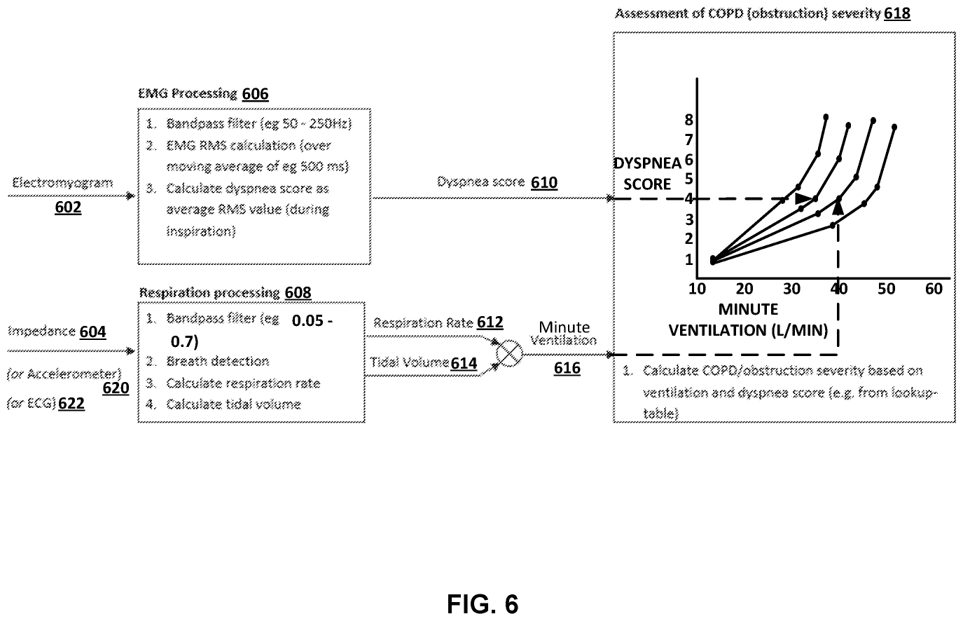

FIG. 6 is a conceptual diagram illustrating an example of determining a measure of COPD severity. For example, IMD 10 may receive or determine EMG signal 602 and receive or determine one or more signals indicative of a respiratory rate and one or more signals indicative of tidal volume, such as impedance measurement 604. In addition to, or alternatively, IMD 10 may receive one or more accelerometer signals 620 and/or an ECG 622, which may be indicative of a respiratory rate and/or a tidal volume of patient 4.

IMD 10 may process EMG 602. For example, IMD 10 may apply a bandpass filter to EMG 602. In some examples, the bandpass filter may be configured to filter out signals outside of a range of 50 Hz to 250 Hz. In some examples, the bandpass filter may be configured to filter out signals outside a range of 100 Hz to 400 Hz. In some examples, IMD 10 may reduce the interference of the cardiac electrical activity (e.g., QRS complexes) in the EMG signal by interpolating the EMG signal during a time interval around the R-wave (e.g., between 80 milliseconds (ms) and 150 ms corresponding to the QRS duration). In some examples, IMD 10 may reduce the interference of the cardiac electrical activity (e.g., QRS complexes) in the EMG signal by adaptive QRS template subtraction, for example, by subtracting a template QRS from the EMG signal during a time interval around the R-wave. In some examples, the template QRS is constructed by averaging a number of QRS complexes. In some examples, IMD 10 may perform a root mean squared (RMS) calculation of the bandpass filtered version of EMG 602 over a predetermined period of time within a range of 0 ms-100 ms, 10 ms-150 ms, or 5 ms 200 ms, such as 100 ms. IMD 10 may calculate a dyspnea score based on an average RMS value during inspiration or inhalation. For example, IMD 10 may use the average RMS value during inspiration as the dyspnea score. In some examples, IMD 10 may use the average of the rectified and bandpass filtered version of EMG 602, for example, during inspiration, to determine the dyspnea score. In some examples, IMD 10 may calculate the area under the curve of the RMS value of the bandpass filtered version of EMG 602, for example, during inspiration, to determine the dyspnea score. In some examples, IMD 10 may use the area under the curve of the rectified and bandpass-filtered version of EMG 602, for example, during inspiration, to determine the dyspnea score. In other examples, IMD 10 may apply a scaling factor to any of the above to determine the dyspnea score, so as to correlate the dyspnea score to a known dyspnea scale (e.g., Borg scale) or to another scale, such as a scale of 1-10.

IMD 10 may process impedance measurement 604 (or additionally, or alternatively, one or more accelerometer signals 620 or ECG 622). For example, IMD 10 may apply a bandpass filter to impedance measurement 604. In some examples, the bandpass filter may be configured to filter out signals outside of a range of 0.05 Hz to 0.7 Hz. In some examples, the bandpass filter may be configured to filter out signals outside a range of 0.01 Hz to 1 Hz. IMD 10 may detect breaths in the bandpass filtered version of the impedance measurement 604 as described above. Based on the detected breaths, IMD 10 may determine respiration rate 612 and tidal volume 614.

IMD 10 may determine minute ventilation 616 based on respiration rate 612 and tidal volume 614. For example, IMD 10 may multiply respiration rate 612 by tidal volume 614 to determine minute ventilation 616. IMD 10 may then determine a measure of COPD severity based on dyspnea score 610 and minute ventilation 616. For example, IMD 10 may determine a ratio of dyspnea score 610 to minute ventilation 616 to determine a measure of COPD severity. In another example, IMD 10 may use dyspnea score 610 and minute ventilation 616 as indices to a lookup table storing measures of COPD severity to determine the measure of COPD severity. For example, referring back to FIG. 5, each of lines 502-508 may represent an “iso-severity” line and areas between each of lines 502-508, as well as areas to the left of line 508 and to the right of line 502 may be assigned a severity level which may be included in the lookup table. In another example, IMD 10 may apply a mathematical formula to dyspnea score 610 and minute ventilation 616 to determine the measure of COPD severity. For example, IMD 10 may utilize a 2-dimensional function (measure of COPD severity=f(EMG, dyspnea) to obtain a continuous variable representing severity level.

FIG. 7 is a composite graphical diagram illustrating examples of lower minute ventilation and higher minute ventilation with a comparable EMG-RMS. Collection of graphs 720 includes a respiration graph 702, filtered EMG graph 706, and EMG-RMS value graph 710. These graphs may collectively be representative of signals which may be received and processed by IMD 10 while monitoring patient 4 during the third period of time discussed with respect to FIG. 5. Collection of graphs 730 includes respiration graph 704, filtered EMG graph 708, and EMG-RMS value graph 712. These graphs may collectively be representative of signals received and processed by IMD 10 while monitoring patient 4 during the second period of time discussed with respect to FIG. 5.

In respiration graphs 702 and 704, the Y axis represents the magnitude of inhaled air measured with a flow meter, each negative deflection being associated with inspiration, and the X axis represents time. While the respiration graphs 702 and 704 were generated using a flow meter, IMD 10 may similarly use impedance measurements to determine respiration, as discussed herein. In this manner, respiration graphs 702 and 704 are representative of signals which may be received and processed by IMD 10 (e.g., each is representative of respiration). In filtered EMG graphs 706 and 708, the Y axis represents EMG magnitude and the X axis represents time. The sharp peaks approximately every second in the filtered EMG graph are the ECG R-peaks, the ‘bursts’ of noise represent the muscle activity of the respiratory muscles during inspiration or inhalation. In EMG-RMS value graphs 710 and 712, the Y axis represents the magnitude of the RMS of the EMG and the X axis represents time.

Comparing respiration graph 702 to respiration graph 704, it can be seen that respiration rates (e.g., when the deflections in the Y axis occur) between the two graphs are comparable, but the deflections in respiration graph 702 are shallower than the deflections in respiration graph 704. Tidal volume may correlate to the area above the downward deflection in respiration graphs 702 and 704. Therefore, the tidal volume shown in respiration graph 702 is lower than the tidal volume shown in respiration graph 704. This means the total ventilation is also lower in respiration graph 702 than in respiration graph 704. Filtered EMG graph 706 and filtered EMG graph 708 are comparable, as are EMG-RMS graph 710 and EMG-RMS graph 712.

IMD 10 may determine a dyspnea score based on EMG-RMS graph 710 and EMG-RMS graph 712. As can be seen in graph 740 point 714 of graph 740 and point 716 of graph 740 both have a dyspnea score of approximately 4. This is because EMG-RMS graph 710 and EMG-RMS graph 712, upon which the dyspnea scores are based, are comparable. However, minute ventilation during the third period of time associated with the dyspnea score of approximately 4 is approximately 35 L/min while minute ventilation during the second period of time associated with the dyspnea score of approximately 4 is approximately 40 L/min. This may indicate that the disease progression of patient 4 between the second period of time and the third period of time has worsened as patient 4 is experiencing an equivalent amount of breathlessness with a lower level of exertion, even though the dyspnea scores are comparable. IMD 10 may determine a measure of COPD severity based on the dyspnea score and the minute ventilation as discussed above which, in some examples, IMD 10 may output to external device 12. In some examples, IMD 10 may output any of respiration graph 702, filtered EMG graph 706, EMG-RMS value graph 710, respiration graph 704, filtered EMG graph 708, EMG-RMS value graph 712 or graph 740 to external device 12.

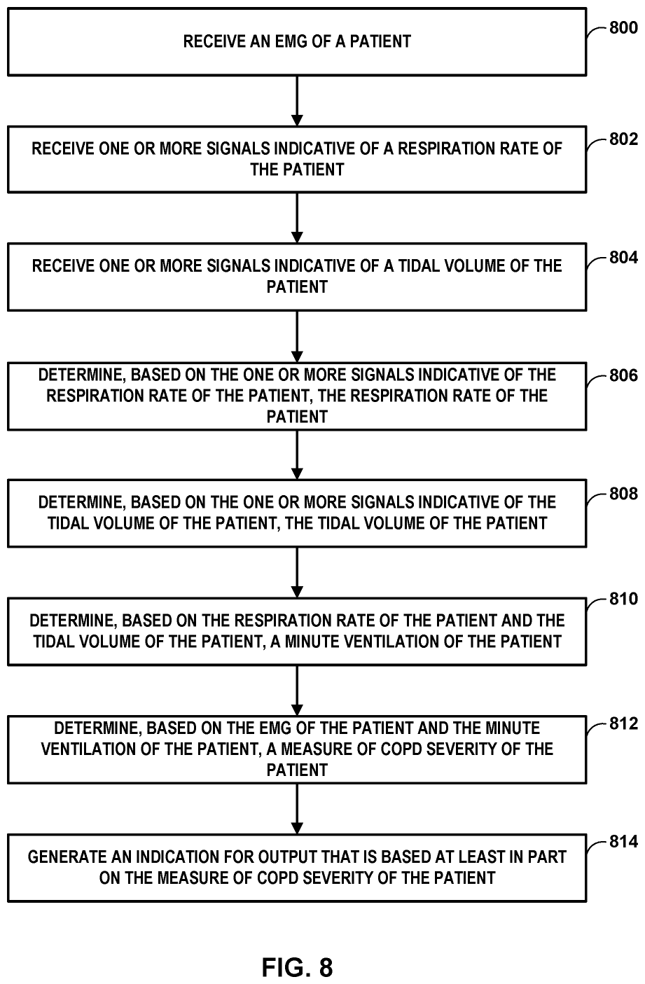

FIG. 8 is a flow diagram illustrating an example of COPD monitoring techniques of this disclosure. Although described as being performed by IMD 10, the example techniques of FIG. 8 may be performed by any one or more devices described herein, e.g., by processing circuitry of any one or more devices described herein, such as IMD 10, external device 12, and computing system 24.

IMD 10 may receive, e.g., sense or measure, an EMG of a patient (800). For example, IMD 10 may sense via sensing circuitry 52, sensors 58, and/or electrodes 16A and 16B (all of FIG. 2) an EMG of patient 4. IMD 10 may receive one or more signals indicative of respiration rate of the patient (802). For example, IMD 10 may apply a current or a voltage to tissue via one or more electrodes and may sense a voltage or current from the tissue via sensing circuitry 52, sensors 58, and/or electrodes 16A and 16B. Alternatively, or additionally, IMD 10 may receive one or more accelerometer signals via sensors 58, which may include an accelerometer. Alternatively, or additionally, IMD 10 may receive an ECG via sensing circuitry 52, sensors 58, and/or electrodes 16A and 16B. Each of such signals may be indicative of a respiration rate of patient 4.

IMD 10 may receive one or more signals indicative of tidal volume of the patient (804). For example, IMD 10 may apply a current or a voltage to tissue via one or more electrodes and may sense a voltage or current from the tissue via sensing circuitry 52, sensors 58, and/or electrodes 16A and 16B and determine an impedance of the tissue. IMD 10 may determine an amplitude (maximum-minimum) of the determined impedance. Alternatively, or additionally, IMD 10 may receive one or more accelerometer signals via sensors 58, which may include an accelerometer. Alternatively, or additionally, IMD 10 may receive an ECG via sensing circuitry 52, sensors 58, and/or electrodes 16A and 16B. Each of such signals may be indicative of a tidal volume of patient 4.

IMD 10 may determine, based on the one or more signals indicative of the respiration rate of the patient, the respiration rate of the patient (806). For example, IMD 10 may perform a breath detection based on the one or more signals indicative of the respiration rate of patient 4. IMD 10 may count a number of breaths during a given time period in the one or more signals indicative of the respiration rate of patient 4 and divide the number of breaths by the given time period to determine the respiration rate of patient 4.

IMD 10 may determine, based on the one or more signals indicative of the tidal volume of the patient, the tidal volume of the patient (808). For example, IMD 10 may perform a breath detection based on the one or more signals indicative of the respiration rate of patient 4. IMD 10 may determine an area above a downward deflection (or an area below an upward deflection) in the one or more signals indicative of the tidal volume of patient 4 to determine the tidal volume of patient 4.

IMD 10 may determine, based on the respiration rate of the patient and the tidal volume of the patient, a minute ventilation of the patient (810). For example, IMD 10 may multiply the determined respiration rate by the determined tidal volume to determine minute ventilation of patient 4.

IMD 10 may determine, based on the minute ventilation of the patient and the EMG of the patient, a measure of COPD severity of the patient (812). For example, IMD 10 may calculate a measure of COPD severity based on the minute ventilation of patient 4 and the EMG of patient 4. For example, IMD 10 may determine a ratio of minute ventilation to the EMG to calculate a measure of COPD severity. IMD 10 may generate an indication for output that is based at least in part on the measure of COPD severity of the patient (814). For example, IMD 10 may generate an indication that may include the measure(s) of COPD severity, physiological parameters of a patient, medical instructions (e.g., instructions for treatment, such as use an inhaler, or to seek medical attention), or the like. In some examples, IMD 10 may output the indication to external device 12 which may, for example, display the indication to be viewed by a clinician, the patient, or a caregiver, thereby facilitating the clinician in assessing the condition of patient and in assessing treatment options for patient 4 or prompting the patient to seek medical treatment. In some examples, the indication may include an alert. External device 12 may output the alert through user interface 406 (FIG. 4), for example. Such an alert may be at least one of visual, auditory, or haptic.

In some examples, the measure of COPD severity comprises a ratio of a value based on the EMG of patient 4 to a value of the minute ventilation of patient 4. For example, the value based on the EMG of patient 4 may be a value of the EMG signal or may be a dyspnea score based on a value of the EMG signal. In some examples, IMD 10 may repeat the method over time to monitor COPD severity over time. In some examples, IMD 10 may determine at least one of the respiration rate of patient 4 or the tidal volume of patient 4 based on at least one of an impedance measurement, one or more accelerometer signals, or an electrocardiogram (ECG).

In some examples, prior to determining the measure of COPD severity of patient 4, IMD 10 may bandpass filter the EMG of patient 4 and determine a root mean square (RMS) of the bandpass filtered EMG of patient 4 over a first predetermined period of time. For example, IMD 10 may use the RMS of the bandpass filtered EMG of patient 4 to determine an EMG metric. For example, IMD 10 may determine the dyspnea score based on the EMG metric. In some examples, IMD 10 may determine a dyspnea score based on at least one of the average of the RMS of the band pass filtered EMG of patient 4, or the area under the curve of the RMS of the band pass filtered EMG of patient 4, over a second predetermined period of time or a predetermined number of inspirations. For example, the second predetermined period of time may be long enough to cover at least one inspiration. In one example, the predetermined period of time may be on the order of 20 seconds. In some examples, IMD 10 may determine an EMG metric as the average of the RMS of the of the band passed filtered EMG of patient 4 over the second predetermined period of time or the predetermined number of inspirations. In the example where IMD 10 determines the average of the RMS of the band pass filtered EMG over a plurality of inspirations, IMD 10 may determine the average of the RMS of the band pass filtered EMG only during inspirations (e.g., excluding expirations). In some examples, IMD 10 may determine an EMG metric as the area under the curve of the RMS of the of the band passed filtered EMG of patient 4 over the second predetermined period of time or the predetermined number of inspirations. In the example, where IMD 10 determines the area under the curve of the RMS of the band pass filtered EMG over a plurality of inspirations, IMD 10 may determine the area under the curve of the RMS of the band pass filtered EMG only during inspirations (e.g., excluding expirations).

In some examples, IMD 10 may determine an EMG metric and determine a dyspnea score. In such examples, the dyspnea score may include at least one of the EMG metric, a processed EMG metric, a score based on data averaged over a representative population, or a score based on patient-specific data of patient 4. For example, IMD 10 may use the EMG metric itself as the dyspnea score for determining the COPD severity of patient 4. In other examples, IMD 10 may determine the dyspnea score by further processing the EMG metric. For example, the relationship between an EMG metric and dyspnea scales, such as the (modified) Borg scale, may be non-linear and IMD 10 may further process the EMG metric to create a more linear relationship between the EMG metric and one or more dyspnea scales. In some examples, the determination of the dyspnea score from the EMG metric may be based on data averaged over a representative population, or from patient-specific data obtained by simultaneously measuring the EMG (and calculating the EMG metric from the EMG) and evaluating the level of dyspnea according to one of the known dyspnea rating scales such as the (modified) Borg scale. In some examples, the relation between the EMG metric and the dyspnea score may be approximated by a quadratic function.

In some examples, prior to determining the measure of COPD severity of patient 4, IMD 10 may bandpass filter at least one of the one or more signals indicative of respiration rate of patient 4 or the one or more signals indicative of tidal volume of patient 4. In some examples, IMD 10 may detect one or more breaths of patient 4, based on the bandpass filtered at least one of the one or more signals indicative of respiration rate of patient 4 or the one or more signals indicative of tidal volume of patient 4.

In some examples, IMD 10 comprises an implantable medical or an insertable medical device, wherein IMD 10 comprises two or more electrodes, the two or more electrodes being oriented towards a core of patient 4, and wherein IMD 10 is implanted or inserted in a patient proximate at least one of rib 6, rib 7, or rib 8.

By generating an indication based at least in part on the measure of COPD severity, a medical device may provide useful information to a clinician in assessing the disease state or disease progression. Such information can be used by the clinician to affect treatment of the patient, thereby reducing the need for hospitalization and/or better preserving the health of the patient.

In some examples, the techniques of the disclosure include a system that comprises means to perform any method described herein. In some examples, the techniques of the disclosure include a computer-readable medium comprising instructions that cause processing circuitry to perform any method described herein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

### The Following Examples are Illustrative of the Techniques Described Herein

Example 1. A medical device system comprising: memory configured to store a measure of COPD severity of a patient; and processing circuitry communicatively coupled to the memory, the processing circuitry being configured to: receive an electromyogram (EMG) of the patient; receive one or more signals indicative of respiration rate of the patient; receive one or more signals indicative of tidal volume of the patient; determine, based on the one or more signals indicative of the respiration rate of the patient, the respiration rate of the patient; determine, based on the one or more signals indicative of the tidal volume of the patient, the tidal volume of the patient; determine, based on the respiration rate of the patient and the tidal volume of the patient, a minute ventilation of the patient; determine, based on the minute ventilation of the patient and the EMG of the patient, the measure of COPD severity of the patient; and generate an indication for output that is based at least in part on the measure of COPD severity of the patient.

Example 2. The medical device system of example 1, wherein the measure of COPD severity of the patient comprises a ratio of a value based on the EMG of the patient to a value of the minute ventilation of the patient.

Example 3. The medical device system of example 1 or example 2, wherein the processing circuitry is further configured to monitor COPD severity over time.

Example 4. The medical device system of any of examples 1-3, wherein as part of determining at least one of the respiration rate of the patient or the tidal volume of the patient, the processing circuitry is configured to determine at least one of the respiration rate of the patient or the tidal volume of the patient based on an impedance measurement, one or more accelerometer signals, or an electrocardiogram (ECG).

Example 5. The medical device system of any of examples 1-4, wherein the processing circuitry is further configured to: prior to determining the measure of COPD severity of the patient, bandpass filter the EMG of the patient; and determine a root mean square (RMS) of the bandpass filtered EMG of the patient over a first predetermined period of time.

Example 6. The medical device system of example 5, wherein as part of determining the measure of COPD severity, the processing circuitry is configured to: determine a dyspnea score based on at least one of the average of the RMS of the band pass filtered EMG of the patient, or the area under the curve of the RMS of the band pass filtered EMG of the patient, over a second predetermined period of time or a predetermined number of inspirations.

Example 7. The medical device system of any of examples 1-6, wherein as part of determining the measure of COPD severity, the processing circuitry is configured to: determine an EMG metric; and determine a dyspnea score, wherein the dyspnea score comprises at least one of the EMG metric, a processed EMG metric, a score based on data averaged over a representative population, or a score based on patient-specific data of the patient.

Example 8. The medical device system of any of examples 1-7, wherein the processing circuitry is further configured to: prior to determining the measure of COPD severity of the patient, bandpass filter at least one of the one or more signals indicative of respiration rate of the patient or the one or more signals indicative of tidal volume of the patient; and detect one or more breaths of the patient, based on at least one of the bandpass filtered one or more signals indicative of respiration rate of the patient or the bandpass filtered one or more signals indicative of tidal volume of the patient.

Example 9. The medical device system of any of examples 1-8, wherein the medical device comprises an implantable medical or an insertable medical device, wherein the medical device comprises two or more electrodes, the two or more electrodes being oriented towards a core of the patient, and wherein the medical device is configured to be implanted or inserted in a patient proximate at least one of rib 6, rib 7, or rib 8.

Example 10. The medical device system of any of examples 1-9, wherein the processing circuitry is further configured to: determine a posture of the patient based on one or more signals from an accelerometer.

Example 11. The medical device system of any of examples 1-10, wherein as part of generating the indication for output, the processing circuitry is configured to at least one of: send a message based at least in part on the measure of COPD severity of the patient to a remote computing device; output for display the indication; output one or more of an audible or haptic indication based at least in part on the measure of COPD severity of the patient; annotate or store data that is based at least in part on the measure of COPD severity of the patient; output a recommendation for change in patient behavior to mitigate COPD severity; or output a recommendation for treatment of the patient to mitigate COPD severity.

Example 12. A method comprising: receiving, by a medical device, an electromyogram (EMG) of a patient; receiving, by the medical device, one or more signals indicative of a respiration rate of the patient; receiving, by the medical device, one or more signals indicative of a tidal volume of the patient; determining, by the medical device and based on the one or more signals indicative of the respiration rate of the patient, the respiration rate of the patient; determining, by the medical device and based on the one or more signals indicative of the tidal volume of the patient, the tidal volume of the patient; determining, by the medical device and based on the respiration rate of the patient and the tidal volume of the patient, a minute ventilation of the patient; determining, by the medical device and based on the minute ventilation of the patient and the EMG of the patient, a measure of COPD severity of the patient; and generating, by the medical device, an indication for output that is based at least in part on the measure of COPD severity of the patient.

Example 13. The method of example 12, wherein the measure of COPD severity of the patient comprises a ratio of a value based on the EMG to a value of the minute ventilation.

Example 14. The method of example 12 or example 13, further comprising repeating the method over time to monitor COPD severity over time.

Example 15. The method of any of examples 12-14, wherein the medical device determines at least one of the respiration rate of the patient or the tidal volume of the patient based on at least one of an impedance measurement, one or more accelerometer signals, or an electrocardiogram (ECG).

Example 16. The method of any of examples 12-15, further comprising: prior to determining the measure of COPD severity of the patient, bandpass filtering the EMG of the patient; and determining a root mean square (RMS) of the bandpass filtered EMG of the patient over a first predetermined period of time.

Example 17. The method of example 16, wherein determining the measure of COPD severity comprises: determining a dyspnea score based on at least one of the average of the RMS of the band pass filtered EMG of the patient, or the area under the curve of the RMS of the band pass filtered EMG of the patient, over a second predetermined period of time or a predetermined number of inspirations.

Example 18. The method of any of examples 12-17, wherein determining the measure of COPD severity comprises: determining an EMG metric; and determining a dyspnea score, wherein the dyspnea score comprises the EMG metric, a processed EMG metric, a score based on data averaged over a representative population, or a score based on patient-specific data of the patient.

Example 19. The method of any of examples 12-18, further comprising: prior to determining the measure of COPD severity of the patient, bandpass filtering at least one of the one or more signals indicative of respiration rate of the patient or the one or more signals indicative of tidal volume of the patient; and detecting one or more breaths of the patient, based on bandpass filtered at least one of the one or more signals indicative of respiration rate of the patient or the one or more signals indicative of tidal volume of the patient.

Example 20. The method of any of examples 12-19, wherein the medical device comprises an implantable medical or an insertable medical device, wherein the medical device comprises two or more electrodes, the two or more electrodes being oriented towards a core of the patient, and wherein the medical device is implanted or inserted in a patient proximate at least one of rib 6, rib 7, or rib 8.

Example 21. The method of claim 12, wherein generating the indication for output comprises at least one of: sending a message based at least in part on the measure of COPD severity of the patient to a remote computing device; outputting for display the indication; outputting one or more of an audible or haptic indication based at least in part on the measure of COPD severity of the patient; annotating or storing data that is based at least in part on the measure of COPD severity of the patient; outputting a recommendation for change in patient behavior to mitigate COPD severity; or outputting a recommendation for treatment of the patient to mitigate COPD severity.

Example 22. A non-transitory computer-readable storage medium storing instructions, which, when executed, cause processing circuitry to: receive an electromyogram (EMG) of a patient; receive one or more signals indicative of respiration rate of the patient; receive one or more signals indicative of tidal volume of the patient; determine, based on the one or more signals indicative of the respiration rate of the patient, the respiration rate of the patient; determine, based on the one or more signals indicative of the tidal volume of the patient, the tidal volume of the patient; determine, based on the respiration rate of the patient and the tidal volume of the patient, a minute ventilation of the patient; determine, based on the minute ventilation of the patient and the EMG of the patient, the measure of COPD severity of the patient; and generate an indication for output that is based at least in part on the measure of COPD severity of the patient.

Various examples have been described. These and other examples are within the scope of the following claims.

What is claimed is:

1. A medical device system comprising:
memory configured to store a measure of COPD severity of a patient; and
processing circuitry communicatively coupled to the memory, the processing circuitry being configured to:
receive an electromyogram (EMG) of the patient;
receive one or more signals indicative of respiration rate of the patient;
receive one or more signals indicative of tidal volume of the patient;
determine, based on the one or more signals indicative of the respiration rate of the patient, the respiration rate of the patient;
determine, based on the one or more signals indicative of the tidal volume of the patient, the tidal volume of the patient;
determine, based on the respiration rate of the patient and the tidal volume of the patient, a minute ventilation of the patient;
determine, based on the minute ventilation of the patient and the EMG of the patient, the measure of COPD severity of the patient; and
generate an indication for output that is based at least in part on the measure of COPD severity of the patient.

2. The medical device system of claim 1, wherein the measure of COPD severity of the patient comprises a ratio of a value based on the EMG of the patient to a value of the minute ventilation of the patient.

3. The medical device system of claim 1, wherein the processing circuitry is further configured to monitor COPD severity over time.

4. The medical device system of claim 1, wherein as part of determining at least one of the respiration rate of the patient or the tidal volume of the patient, the processing circuitry is configured to determine at least one of the respiration rate of the patient or the tidal volume of the patient based on an impedance measurement, one or more accelerometer signals, or an electrocardiogram (ECG).

5. The medical device system of claim 1, wherein the processing circuitry is further configured to:
prior to determining the measure of COPD severity of the patient, bandpass filter the EMG of the patient; and
determine a root mean square (RMS) of the bandpass filtered EMG of the patient over a first predetermined period of time.

6. The medical device system of claim 5, wherein as part of determining the measure of COPD severity, the processing circuitry is configured to:
determine a dyspnea score based on at least one of the average of the RMS of the band pass filtered EMG of the patient, or the area under the curve of the RMS of the band pass filtered EMG of the patient, over a second predetermined period of time or a predetermined number of inspirations.

7. The medical device system of claim 1, wherein as part of determining the measure of COPD severity, the processing circuitry is configured to:
determine an EMG metric; and
determine a dyspnea score, wherein the dyspnea score comprises at least one of the EMG metric, a processed EMG metric, a score based on data averaged over a representative population, or a score based on patient-specific data of the patient.

8. The medical device system of claim 1, wherein the processing circuitry is further configured to:
prior to determining the measure of COPD severity of the patient, bandpass filter at least one of the one or more signals indicative of respiration rate of the patient or the one or more signals indicative of tidal volume of the patient; and
detect one or more breaths of the patient, based on at least one of the bandpass filtered one or more signals indicative of respiration rate of the patient or the bandpass filtered one or more signals indicative of tidal volume of the patient.

9. The medical device system of claim 1, wherein the medical device system comprise a medical device, the medical device being configured to be implanted or inserted in a patient proximate at least one of rib 6, rib 7, or rib 8 and wherein the medical device system comprises two or more electrodes, the two or more electrodes being oriented towards a core of the patient.

10. The medical device system of claim 1, wherein the processing circuitry is further configured to:
determine a posture of the patient based on one or more signals from an accelerometer.

11. The medical device system of claim 1, wherein as part of generating the indication for output, the processing circuitry is configured to at least one of:
send a message based at least in part on the measure of COPD severity of the patient to a remote computing device;
output for display the indication;
output one or more of an audible or haptic indication based at least in part on the measure of COPD severity of the patient;
annotate or store data that is based at least in part on the measure of COPD severity of the patient;
output a recommendation for change in patient behavior to mitigate COPD severity; or
output a recommendation for treatment of the patient to mitigate COPD severity.

12. A method comprising:
receiving, by a medical device, an electromyogram (EMG) of a patient;
receiving, by the medical device, one or more signals indicative of a respiration rate of the patient;
receiving, by the medical device, one or more signals indicative of a tidal volume of the patient;
determining, by the medical device and based on the one or more signals indicative of the respiration rate of the patient, the respiration rate of the patient;
determining, by the medical device and based on the one or more signals indicative of the tidal volume of the patient, the tidal volume of the patient;
determining, by the medical device and based on the respiration rate of the patient and the tidal volume of the patient, a minute ventilation of the patient;
determining, by the medical device and based on the minute ventilation of the patient and the EMG of the patient, a measure of COPD severity of the patient; and
generating, by the medical device, an indication for output that is based at least in part on the measure of COPD severity of the patient.

13. The method of claim 12, wherein the measure of COPD severity of the patient comprises a ratio of a value based on the EMG to a value of the minute ventilation.

14. The method of claim 12, further comprising repeating the method over time to monitor COPD severity over time.

15. The method of claim 12, wherein the medical device determines at least one of the respiration rate of the patient or the tidal volume of the patient based on at least one of an impedance measurement, one or more accelerometer signals, or an electrocardiogram (ECG).

16. The method of claim 12, further comprising:
prior to determining the measure of COPD severity of the patient, bandpass filtering the EMG of the patient; and
determining a root mean square (RMS) of the bandpass filtered EMG of the patient over a first predetermined period of time.

17. The method of claim 16, wherein determining the measure of COPD severity comprises:
determining a dyspnea score based on at least one of the average of the RMS of the band pass filtered EMG of the patient, or the area under the curve of the RMS of the band pass filtered EMG of the patient, over a second predetermined period of time or a predetermined number of inspirations.

18. The method of claim 12, wherein determining the measure of COPD severity comprises:
determining an EMG metric; and
determining a dyspnea score, wherein the dyspnea score comprises at least one of the EMG metric, a processed EMG metric, a score based on data averaged over a representative population, or a score based on patient-specific data of the patient.

19. The method of claim 12, further comprising:
prior to determining the measure of COPD severity of the patient, bandpass filtering at least one of the one or more signals indicative of respiration rate of the patient or the one or more signals indicative of tidal volume of the patient; and
detecting one or more breaths of the patient, based on bandpass filtered at least one of the one or more signals indicative of respiration rate of the patient or the one or more signals indicative of tidal volume of the patient.

20. A non-transitory computer-readable storage medium storing instructions, which, when executed, cause processing circuitry to:
receive an electromyogram (EMG) of a patient;
receive one or more signals indicative of respiration rate of the patient;
receive one or more signals indicative of tidal volume of the patient;
determine, based on the one or more signals indicative of the respiration rate of the patient, the respiration rate of the patient;
determine, based on the one or more signals indicative of the tidal volume of the patient, the tidal volume of the patient;
determine, based on the respiration rate of the patient and the tidal volume of the patient, a minute ventilation of the patient;
determine, based on the minute ventilation of the patient and the EMG of the patient, a measure of COPD severity of the patient; and
generate an indication for output that is based at least in part on the measure of COPD severity of the patient.
